(19) United States
(12) Patent Application Publication
(10) Pub. No.: US 2021/0038566 A1
(43) Pub. Date: Feb. 11, 2021

(54) USE OF MODULATORS OF NEET PROTEINS FOR THE TREATMENT OF INFECTION

(71) Applicant: ENYO PHARMA, LYON (FR)

(72) Inventors: ERIC MELDRUM, RIEHEN (CH); BENOÎT DE CHASSEY, LYON (FR)

(21) Appl. No.: 16/967,752

(22) PCT Filed: Feb. 8, 2019
(86) PCT No.: PCT/EP2019/053083

§ 371 Date: Aug. 6, 2020

(30) Foreign Application Priority Data
Feb. 8, 2018 (EP) 18305135.8

(51) Int. Cl. A61K 31/381; A61K 31/451; A61K 31/4439; A61K 45/06; A61P 31/16

(52) U.S. Cl. CPC A61K 31/381; A61K 31/451; A61P 31/16; A61K 45/06; A61K 31/4439

(57) ABSTRACT

The present invention relates to the use of modulators of NEET proteins for the treatment of infection, in particular viral or bacterial infection.

### FIELD OF THE INVENTION

The present invention relates to the field of medicine, in particular drugs against infection, especially antiviral or antibacterial drugs.

### BACKGROUND OF THE INVENTION

Viruses are small infectious agents that replicate only inside living cells of other organisms. They can infect all types of life forms, from animals and plants to microorganisms, including bacteria and archaea. Among them, more than 400 species of virus are known to be responsible for diseases in humans, many of them leading to serious pathologies and eventually death. In particular, HIV was classified as the sixth leading cause of death worldwide in 2012 with 1.5 millions of deaths per year (WHO, Fact sheet No 310, 2014). Seasonal influenza viruses are responsible for flu that affects approximately 20% of the world population and causes 250,000 to 500,000 deaths per year (WHO, Fact sheet No 211, 2014). Among other examples, Hepatitis B and C are responsible altogether for about 1.4 million deaths each year and human Papillomaviruses are responsible of cervix cancer, the second most common female cancer worldwide, leading to 270,000 deaths in 2012 (WHO, Fact sheets, 2016).

Because viruses use vital metabolic pathways within host cells to replicate, they are difficult to eliminate without using drugs that cause toxic effects to host cells in general. The most effective medical approaches to viral diseases are vaccinations to provide immunity to infection, and antiviral drugs that selectively interfere with viral replication. Vaccines are very effective on stable viruses for a preventive use. However, vaccines are of limited use in treating a patient who has already been infected. They are also difficult to successfully deploy against rapidly mutating viruses, such as influenza (the vaccine for which is updated every year) and HIV. Antiviral drugs may be particularly useful in these cases.

Antiviral drugs are a class of medication used specifically for treating viral infections. Antiviral drugs do not destroy their target pathogens, instead they inhibit their development. Antiviral drugs may target any stage of the viral life cycle: attachment to a host cell, uncoating, replication and expression of the viral genome, assembly of viral components into complete viral particles, and release of viral particles to infect new host cells. The most common antiviral drugs are nucleoside analogues that block viral replication. Most antiviral drugs are used for specific viral infections, while broad-spectrum antiviral drugs are effective against a wide range of viruses.

Soon after the development of antiviral drugs, resistance appeared. Antiviral drug resistance can be defined as a decreased susceptibility to a drug through either a minimally effective, or completely ineffective, treatment response to prevent associated illnesses from a particular virus. Antiviral drug resistance remains a major obstacle to antiviral therapy as it has developed to almost all specific and effective antiviral drugs. For example, there are two main groups of antiviral drugs available for treatment and prophylaxis of influenza: M2 inhibitors (amantadine and rimantadine) and neuraminidase inhibitors (oseltamivir and zanamivir). Despite the effectiveness of these drugs in reducing influenza-related morbidity and mortality, the emergence of drug resistance poses a critical limitation on their application and have raised an urgent need for developing new anti-influenza drugs against resistant forms.

Thus, there is a strong need for the development of new antiviral drugs, and in particular broad-spectrum antiviral drugs. The present invention seeks to meet these and other needs.

### SUMMARY OF THE INVENTION

The present invention relates to the identification of NEET proteins as targets for developing a new class of antimicrobial agents, especially antiviral or antibacterial drugs.

The present invention relates to a modulator of a NEET protein for use for treating an infectious disease.

In a first embodiment, the infectious disease is a viral infection. Optionally, the viral infection is an infection by a virus selected from the group consisting of Alphaviridae, Flaviviridae, Hepadnaviridae, Herpesviridae, Orthomyxoviridae, Papovaviridae, Paramyxoviridae, Picornaviridae, Polyomaviridae, Reoviridae, Retroviridae, Rhabdoviridae, and Tobamoviruses.

More specifically, the virus is selected from the group consisting of
Barmah Forest virus, Middelburg virus, Ndumu virus, Bebaru virus, Chikungunya virus, Mayaro virus, O'nyong'nyong virus, Ross River virus, Semliki Forest virus, Sindbis virus, Una virus, Eastern equine encephalitis virus, Tonate virus, Venezuelan equine encephalitis virus, Cabassou virus, Everglades virus, Mosso das Pedras virus, Mucambo virus, Parmana virus, Pixuna virus, Rio Negro virus, Trocara virus, Aura virus, Babanki virus, Kyzylagach virus, Ockelbo virus, Whataroa virus, Sleeping disease virus, Samon pancreatic disease virus, Southern elephant seal virus, and Western equine encephalitis virus; preferably selected from the group consisting of Barmah Forest virus, Chikungunya virus, Mayaro virus, O'nyong'nyong virus, Ross Rivervirus, Semliki Forest virus, Sindbis virus, Una virus, Eastern equine encephalitis virus, Tonate virus, Venezuelan equine encephalitis virus and Western equine encephalitis virus;
dengue virus, Hepatitis C virus, Japanese encephalitis virus, West Nile virus, yellow fever virus, Zika virus, Tick-borne encephalitis virus, Kyasanur forest disease virus, Murray Valley encephalitis virus, and Saint Louis encephalitis virus;
Hepatitis B virus;
Herpes Simplex virus 1 (HSV-1), Herpes Simplex virus 2 (HSV-2), Varicella zoster virus (VZV), Epstein-Barr virus (EBV), Cytomegalovirus (CMV), Roseolovirus (HHV-6A and 6B), HHV-7 and Kaposi's sarcoma-associated herpesvirus (KSHV);
Influenza virus A, Influenza virus B, Influenza virus C, Isavirus, Thogotovirus and Quaranjavirus, preferably selected from the group consisting of Influenza virus A and Influenza virus B, for instance selected from the subtypes consisting of H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7;
Papillomavirus (HPC) and Polyomavirus, especially Simian virus 40, Merkel cell polyomavirus, Trichodysplasia spinulosa polyomavirus, BK polyomavirus, JC polyomavirus and Human polyomavirus 7;
Rubulavirus, Morbillivirus, Pneumovirus, Metapneumovirus, Avulavirus, Ferlavirus, Henipavirus, Respirovirus, preferably from the group consisting of the mumps virus, measles virus, human parainfluenza viruses (HPIV), especially HPIV-1, HPIV-2, HPIV-3 or HPIV-4, respiratory syncytial virus (RSV), in particular Human respiratory syncytial virus (HRSV), canine distemper virus, phocine distemper virus, cetacean morbillivirus, Newcastle disease virus, rinderpest virus, Hendra birus and Nipah virus;
Aphthovirus, Aquamavirus, Avihepatovirus, Cardiovirus, Cosavirus, Dicipivirus, Enterovirus, Erbovirus, Hepatovirus, Kobuvirus, Megrivirus, Parechovirus, Piscevirus, Rhinovirus, Salivirus, Sapelovirus, Senecavirus, Techovirus, and Tremovirus, in a particular embodiment, a Rhinovirus, for instance a Rhinovirus A, Rhinovirus B or Rhinovirus C;
Alpharetrovirus; especially Avian leukosis virus and Rous sarcoma virus; Betaretrovirus, especially Mouse mammary tumour virus; Gammaretrovirus, especially Murine leukemia virus and Feline leukemia virus; Deltaretrovirus, especially Bovine leukemia virus and Human T-lymphotropic virus; Epsilonretrovirus, especially Walleye dermal sarcoma virus; Lentivirus, especially Human immunodeficiency virus 1 and Simian, Feline immunodeficiency viruses; Spumavirus, especially Simian foamy virus; and,
vesiculovirus, especially vesicular stomatitis virus, lyssavirus, espcially rabies virus, Ephemerovirus, novirhabdovirus, cytorhabdovirus and nucleorhabdovirus.

In a second embodiment, the infectious disease is a bacterial infection. Optionally, the infectious disease can be an infection by a bacterium selected from the group consisting of Helicobacter pylori, Burkholderia cepacia, Pseudomonas aeruginosa, Pseudomonas fluorescens, Pseudomonas acidovorans, Pseudomonas alcaligenes, Pseudomonas putida, Stenotrophomonas maltophilia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella parapertussis, Bordetella bronchiseptica, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Campylobacter fetus, Campylobacter jejuni, Campylobacter coli, Borrelia burgdorferi, Vibrio cholerae, Vibrio parahaemolyticus, Listeria monocytogenes, Neisseria gonorrhoeae, Neisseria meningitidis, Kingella denitrificans, Kingella indologenes, Kingella kingae, Kingella oralis, Legionella pneumophila, Moraxella bovis, Moraxella catarrhalis, Moraxella lacunata, Gardnerella vaginalis, Bacteroides fragilis, Bacteroides distasonis, Bacteroides vulgatus, Bacteroides ovalus, Bacteroides thetaiotaomicron, Bacteroides uniformis, Bacteroides eggerthii, Bacteroides splanchnicus, Clostridium difficile, Clostridium tetani, Mycobacterium species, Corynebacterium ulcerans, Streptococcus agalactiae, Gardnerella vaginitis, Streptococcus pyogenes, Enterococcus faecalis, Enterococcus faecium, Fusobacterium nucleatum, Porphyromonas gingivalis, Vibrio vulnificus, Clostridium botulinum, Corynebacterium diptheriae, Staphylococcus aureus, Staphylococcus epidermidis, Staphylococcus saprophyticus, Staphylococcus intermedius, Staphylococcus hyicus, Staphylococcus haemolyticus, Staphylococcus hominis, and Staphylococcus saccharolyticus. More specifically, the bacterial infection can be an infection by a Mycobacterium species, such as M. africanum, M. bovis, M. bovis BCG, M. canetti, M. caprae, M. microti, M. mungi, M. orygis, M. pinnipedii, M. suricattae, M. tuberculosis, M. avium, M. avium paratuberculosis, M. avium silvaticum, M. avium “hominissuis”, M. colombiense, M. indicus pranii, M. asiaticum, M. gordonae, M. gastri and M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. bohemicum, M. botniense, M. branderi, M. celatum, M. chimaera, M. conspicuum, M. cookie, M. doricum, M. farcinogenes, M. haemophilum, M. heckeshornense, M. intracellular, M. lacus, M. leprae, M. lepraemurium, M. lepromatosis, M. liflandii, M. malmoense, M. marinum, M. monacense, M. montefiorense, M. murale, M. nebraskense, M. saskatchewanense, M. scrofulaceum, M. shimoidei, M. szulgai, M. tusciae, M. xenopi, M. yongonense, M. intermedium, M. abscessus, M. chelonae, M. bolletii, M. fortuitum, M. fortuitum subsp. Acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodie, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense, and M. tokaiense, preferably Mycobacterium tuberculosis, Mycobacterium leprae, or Mycobacterium ulcerans.

The NEET protein modulator can be selected in the group consisting of a molecular compound and/or a small molecule, and/or a miRNA and/or a siRNA, and/or a mitoNEET CRISPR/Cas9 KO Plasmid, and/or antisense oligonucleotides and/or an antibody.

Preferably, the NEET protein modulator is selected among the group consisting of Magnolol, 3,3/-di-L-tyrosine, Ac—NH-3,3′-di-L-Tyr-CO—NH2, Ac—NH-3,3′-di-L-Tyr-Gly-Gly-CO—NH2, Ac—NH-3,3′-di-L-Tyr-Ala-Ala-CO—NH, Ac—NH-3,3′-di-L-Tyr-R1-R2-6CO—NH2, Enterobactin, Cromolyn, Quercetin, Naringenin, (−)-Epicatechin, Procyanidin A2, Tran Resvertrol, Epsilon-Viniferin, Laetevirenol A, NL-1, NL-2, NL-3, NL-4, NL-5, NL-6, NL-7, NL-8, NL-9, NL-10, NL-11, N L-12, NL-14, N L-14, N L-15, NL-15, NL-16, N L-17, N L-18, NL-19, N L-20, NL-21, Resveratrol 3-sulfate, Curcumin, Kaempferol, NL-23, NL-24, NL-25, NL-26, NL-27, NL-28, NL-31, NL-32, AG104, NL-33, NL-34, Furosemide, alpha-Hydro-cinnamic acid, Glibenclamide, 7917584, 6209863, 4-Amino-1,8-naphthalimide, Triapine, Nitrofurantoin, Dantrolene, CCCP, 6636424, 6373721, 7320244, 5472855, 6634507, 5119666, 7138125, 7722368, 5472855, GSK-LSD1, Tryprostatin-A, Doxorubicin, Ursodiol, Gemfibrozil, Thiazolidinedione, Thiazolidinedione salt, Thiazolidinedione derivatives, Pioglitazone, Rosiglitazone, Rivoglitazone, Troglitazone, MSDC-0160, MSDC-0602, TT01001, MAD-28 and Resveratrol.

In one embodiment, the NEET protein modulator is a thiazolidinedione (TZD) derivative, in particular selected from the group consisting of pioglitazone, rosiglitazone, troglitazone, MSDC-0160, MSDC-0602, TZD NL-1, resveratrol, resveratrol-3-sulfate, TT01001 and MAD-28.

In a preferred embodiment, the NEET protein modulator is a thiazolidinedione (TZD), salt and/or derivative thereof, preferably selected in the group consisting of pioglitazone, rosiglitazone, Rivoglitazone, troglitazone, MSDC-0160, MSDC-0602, and NL-1.

Optionally, the NEET protein modulator is used in combination with another active ingredient, such as an antiviral agent or an antibacterial agent.

### DETAILED DESCRIPTION OF THE INVENTION

### Definition

As used herein, the terms “treatment”, “treat” or “treating” refer to any act intended to ameliorate the health status of patients such as therapy, prevention, prophylaxis and retardation of a disease, in particular an infection, preferably a viral infection. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human, including adult, child, newborn and human at the prenatal stage. However, the term “subject” can also refer to non-human animals, in particular mammals such as dogs, cats, horses, cows, pigs, sheep and non-human primates, among others.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refers to a component of a pharmaceutical composition having a therapeutic effect.

As used herein, the term “therapeutic effect” refers to an effect induced by an active ingredient, or a pharmaceutical composition according to the invention, capable to prevent or to delay the appearance of a disease, such as an infection, preferably a viral infection, or to cure or to attenuate the effects of a disease.

As used herein, the term “effective amount” refers to a quantity of an active ingredient or of a pharmaceutical composition which prevents, removes or reduces the deleterious effects of the disease, particularly infectious disease. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

The term “modulator”, as used herein, refers to a molecule, a chemical or a substance targeting, added, applied or active to another, to modulate a reaction or to prevent an unwanted change. As used herein, the term “modulator” refer to any molecule or compound targeting and/or binding specifically NEET proteins.

The term “modulator”, as used herein, refers to a molecule, a chemical or a substance targeting, added, applied or active to another, to slowdown or inhibit a reaction or to prevent an unwanted change. As used herein, the term “modulator” refer to any molecule or compound having an effect on Fe—S cluster binding by the NEET protein. The “modulator” as used herein may be either a stabiliser or a destabiliser. The term “stabiliser” as used herein refers to any compound, chemical, or substance able to stabilize the Fe—S cluster binding the NEET protein. Particularly, a stabiliser reduces the off-rate of iron (Fe) or slows the release of bound Fe—S. In a preferred embodiment, a modulator may be a “stabiliser” when it is able to increase the time needed to reach 50% Fe—S cluster bound loss by more than 25%. The term “destabiliser” as used herein refers to any compound, chemical, or substance able to destabilize the Fe—S cluster binding the NEET protein. Particularly, a destabiliser enhances the off-rate of iron (Fe). In a preferred embodiment, a modulator may be a “destabiliser” when it is able to decrease the time needed to reach 50% Fe—S cluster bound loss by more than 25%. The effect of the modulator can be determined by the protocol as follows. NEET protein/2Fe-2S cluster stability can be assessed by monitoring the decay in absorbance of its characteristic 458-nm peak (characteristic of the oxidized 2Fe-2S cluster) over time. Each NEET protein (mitoNEET, NAF-1 and Miner 2) can be tested for its Fe—S binding capacities in the absence or presence of the modulator. The rate of cluster release (time in minutes to achieve 50% loss of bound Fe—S cluster) is compared for each NEET protein in the presence of the modulator (in a 1:3 protein:modulator molar ratio) relative to each protein alone. At pH 6, all the three NEET proteins (mitoNEET, NAF-1 and Miner 2) have a characteristic rate of loss of the bound Fe—S cluster that can be measured by the decrease of absorbance at wavelength 458 nm over time, using a spectrophotometer. Thus, Bis-Tris buffer (100 mM Bis-Tris pH6, 100 mM Nacl) is used at pH 6 to dilute either DMSO (Blank sample: Bis-Tris Buffer pH 6, 66 μM DMSO), DMSO and one of the three NEET proteins (Control sample: Bis-Tris Buffer pH 6, 66 μM DMSO, 20 μM purified NEET protein) or DMSO, one of the three NEET proteins and a modulator (Test sample: Bis-Tris Buffer pH 6, 66 μM DMSO, 20 μM purified NEET protein, 60 μM compound of the invention). A reaction mix containing DMSO diluted in the Bis-Tris Buffer with or without a compound of the invention was prepared. The purified NEET protein is the last component added to the reaction mix which was then aliquoted into 4 replicates in 96 wells plates. The absorbance at wavelength 458 nm is taken at 5 minutes intervals at 37° C. with a spectrofluorimeter. The assay run time for CISD2 gene product (NAF-1) is 500 minutes and, 180 minutes for both the CISD 1 gene product (mitoNEET) and the CISD3 gene product (Miner 2). As used herein, CISD and NEET can be replaced by each other.

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the actives ingredients.

The term “infectious disease”, as used herein, refers to a disease resulting from the presence and/or activity of a pathogenic agent, e.g. microbial agent such as bacteria and/or viruses.

As used herein, the term “viral infection” refers to the invasion of an organism's body tissues by disease-causing viruses, their multiplication, and the reaction of host tissues to these viruses.

The terms “viral agent”, “viral pathogen” and “disease-causing virus”, as used herein, are equivalent and refer to viruses that cause infection.

As used herein, the terms “antiviral”, “antiviral molecule”, “antiviral drug” or “antiviral agent” are equivalent and refer to a molecule used in the treatment and prevention of viral infections. Antiviral drugs do not destroy their target viruses, instead they inhibit their development. Antiviral drugs may target any stage of the viral life cycle, in particular attachment to a host cell, release of viral genes and possibly enzymes into the host cell, replication of viral components using host-cell machinery, assembly of viral components into complete viral particles, or release of viral particles to infect new host cells.

As used herein, the term “bacterial infection” refers to the invasion of an organism's body tissues by disease-causing bacteria, their multiplication, and the reaction of host tissues to these bacteria.

The terms “bacterial agent” and “bacterial pathogen” as used herein, are equivalent and refer to bacteria that cause infection.

As used herein, the terms “antibacterial”, “antibacterial molecule”, “antibacterial drug” or “antibacterial agent” are equivalent and refer to a molecule used in the treatment and prevention of bacterial infections. Antibacterial drugs or antibiotics destroy their target bacteria or inhibit their development.

### Use of Modulators of NEET Proteins

The invention relates to a modulator of a NEET protein for use in the treatment of an infectious disease, preferably a viral infection or a bacterial infection. The present invention relates to a method for treating an infectious disease, preferably a viral infection or a bacterial infection, in a subject, wherein a therapeutically effective amount of a modulator of a NEET protein is administered to said subject suffering of an infectious disease, preferably a viral infection or a bacterial infection. The present invention relates to the use of a modulator of a NEET protein as an anti-infectious agent, preferably an antiviral agent or an antibacterial agent. The invention also relates to the use of a modulator of a NEET protein for the manufacture of a medicine for the treatment of an infectious disease, preferably a viral infection or a bacterial infection.

The present invention also relates to the use of a modulator of a NEET protein as a phytosanitary agent and a phytosanitary composition comprising a modulator of a NEET protein. It further relates to a method for treating a plant against infection, especially infection by a virus or a bacterium, comprising contacting the plant with an efficient amount of a modulator of a NEET protein.

The present invention also relates to the use of a modulator of a NEET protein as a research tool for studying viral infections. It further relates to a method for blocking viral infection in a cell, a tissue or a subject using a modulator of a NEET protein.

The viral agent can be a DNA virus or an RNA virus. The viral agent can be selected from the group consisting of Alphaviridae, Flaviviridae, Hepadnaviridae, Herpesviridae, Orthomyxoviridae, Papovaviridae, Paramyxoviridae, Picornaviridae, Polyomaviridae, Reoviridae, Retroviridae, Rhabdoviridae, and Tobamoviruses.

In one embodiment, the Alphaviridae is selected from the group consisting of Barmah Forest virus, Middelburg virus, Ndumu virus, Bebaru virus, Chikungunya virus, Mayaro virus, O'nyong'nyong virus, Ross River virus, Semliki Forest virus, Sindbis virus, Una virus, Eastern equine encephalitis virus, Tonate virus, Venezuelan equine encephalitis virus, Cabassou virus, Everglades virus, Mosso das Pedras virus, Mucambo virus, Parmana virus, Pixuna virus, Rio Negro virus, Trocara virus, Aura virus, Babanki virus, Kyzylagach virus, Ockelbo virus, Whataroa virus, Sleeping disease virus, Samon pancreatic disease virus, Southern elephant seal virus, and Western equine encephalitis virus; preferably selected from the group consisting of Barmah Forest virus, Chikungunya virus, Mayaro virus, O'nyong'nyong virus, Ross River virus, Semliki Forest virus, Sindbis virus, Una virus, Eastern equine encephalitis virus, Tonate virus, Venezuelan equine encephalitis virus and Western equine encephalitis virus.

In one embodiment, the Flaviviridae is selected from the group consisting of dengue virus, Hepatitis C virus, Japanese encephalitis virus, West Nile virus, yellow fever virus, Zika virus, Tick-borne encephalitis virus, Kyasanur forest disease virus, Murray Valley encephalitis virus, and Saint Louis encephalitis virus.

In one embodiment, the Hepadnaviridae is selected from the group consisting of Hepatitis B virus.

In one embodiment, the Herpesviridae is selected from the group consisting of Herpes Simplex virus 1 (HSV-1), Herpes Simplex virus 2 (HSV-2), Varicella zoster virus (VZV), Epstein-Barr virus (EBV), Cytomegalovirus (CMV), Roseolovirus (HHV-6A and 6B), HHV-7 and Kaposi's sarcoma-associated herpesvirus (KSHV).

In one embodiment, the Orthomyxoviridae is selected from the group consisting of Influenza virus A, Influenza virus B, Influenza virus C, Isavirus, Thogotovirus and Quaranjavirus, preferably selected from the group consisting of Influenza virus A and Influenza virus B. In one embodiment, the Influenza virus A is selected from the subtypes consisting of H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7.

In one embodiment, the Papovaviridae is selected from the group consisting of Papillomavirus (HPC) and Polyomavirus, especially Simian virus 40, Merkel cell polyomavirus, Trichodysplasia spinulosa polyomavirus, BK polyomavirus, JC polyomavirus and Human polyomavirus 7.

In one embodiment, the Paramyxoviridae is selected from the group consisting of Rubulavirus, Morbillivirus, Pneumovirus, Metapneumovirus, Avulavirus, Ferlavirus, Henipavirus, Respirovirus. In a particular embodiment, the Paramyxoviridae is the mumps virus, measles virus, human parainfluenza viruses (HPIV), especially HPIV-1, HPIV-2, HPIV-3 or HPIV-4, respiratory syncytial virus (RSV), in particular Human respiratory syncytial virus (HRSV), canine distemper virus, phocine distemper virus, cetacean morbillivirus, Newcastle disease virus, rinderpest virus, Hendra birus and Nipah virus.

In one embodiment, the Picornaviridae is selected from the group consisting of Aphthovirus, Aquamavirus, Avihepatovirus, Cardiovirus, Cosavirus, Dicipivirus, Enterovirus, Erbovirus, Hepatovirus, Kobuvirus, Megrivirus, Parechovirus, Piscevirus, Rhinovirus, Salivirus, Sapelovirus, Senecavirus, Techovirus, and Tremovirus. In a particular embodiment, the Picornaviridae is a Rhinovirus, for instance a Rhinovirus A, Rhinovirus B or Rhinovirus C.

In one embodiment, the Retroviridae is selected from the group consisting of Alpharetrovirus; especially Avian leukosis virus and Rous sarcoma virus; Betaretrovirus, especially Mouse mammary tumour virus; Gammaretrovirus, especially Murine leukemia virus and Feline leukemia virus; Deltaretrovirus, especially Bovine leukemia virus and Human T-lymphotropic virus; Epsilonretrovirus, especially Walleye dermal sarcoma virus; Lentivirus, especially Human immunodeficiency virus 1 and Simian, Feline immunodeficiency viruses; Spumavirus, especially Simian foamy virus.

In one embodiment, the Rhabdoviridae is selected from the group consisting of vesiculovirus, especially vesicular stomatitis virus, lyssavirus, especially rabies virus, Ephemerovirus, novirhabdovirus, cytorhabdovirus and nucleorhabdovirus.

In one preferred embodiment, the viral agent according to the invention is selected from the group consisting in Herpesviridae such as Varicella zoster virus (VZV), Epstein-Barr (EB) virus, Herpes simplex virus of type 1 (HSV-1), Kaposis sarcoma herpesvirus (KSHV), murine γ-HV68 virus (γ-MHV68), or human cytomegalovirus (HCMV); Hepadnaviridae such as Hepatitis virus B (HBV); Papovaviridae such as Human papillomavirus type 16 (HPV16); Parvoviridae such as Human parvovirus B19; Polyomaviridae such as Simian virus 40; Retroviridae such has Human immunodeficiency virus 1 (HIV-1), or Simian immunodeficiency virus type 1 (SIV 1); Orthomyxoviridae such as Influenza A virus; Flaviviridae such as Dengue virus, or Hepatitis C virus; Picornaviridae such as Poliovirus, Coxsakievirus B3 (CVB3), or Coxsakievirus B4 (CVB4); Reoviridae such as Rotavirus; Alphaviridae such as Sindbis virus; Tobamoviruses such as Tabacco mosaic virus; Rhabdoviridae such as vesicular stomatitis virus. More preferably, the viral agent according to the invention is an influenza virus. Still preferably, the viral agent according to the invention is an influenza virus A or B, even more preferably an influenza virus A.

In another preferred embodiment, the viral agent according to the invention presents an antiviral resistance to classic antiviral drugs. The terms “antiviral resistance”, “antiviral agent resistance” or “antiviral drug resistance”, as used herein, are equivalent and refer to the ability of viruses to resist the effects of an antiviral agent previously used to treat them.

Antiviral resistance can be defined by a decreased susceptibility to a drug through either a minimally effective, or completely ineffective, treatment response to prevent associated illnesses from a particular virus.

The bacterium can be gram-negative and gram-positive bacteria, preferably an infectious bacterium. Such gram-positive bacteria include, but are not limited to, Pasteurella species, Staphylococci species, and Streptococcus species.

Specific examples of bacteria include but are not limited to, Helicobacter pylori, Burkholderia cepacia, Pseudomonas aeruginosa, Pseudomonas fluorescens, Pseudomonas acidovorans, Pseudomonas alcaligenes, Pseudomonas putida, Stenotrophomonas maltophilia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella parapertussis, Bordetella bronchiseptica, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Campylobacter fetus, Campylobacter jejuni, Campylobacter coli, Borrelia burgdorferi, Vibrio cholerae, Vibrio parahaemolyticus, Listeria monocytogenes, Neisseria gonorrhoeae, Neisseria meningitidis, Kingella denitrificans, Kingella indologenes, Kingella kingae, Kingella oralis, Legionella pneumophila, Moraxella bovis, Moraxella catarrhalis, Moraxella lacunata, Gardnerella vaginalis, Bacteroides fragilis, Bacteroides distasonis, Bacteroides vulgatus, Bacteroides ovalus, Bacteroides thetaiotaomicron, Bacteroides uniformis, Bacteroides eggerthii, Bacteroides splanchnicus, Clostridium difficile, Clostridium tetani, Mycobacterium species, Corynebacterium ulcerans, Streptococcus agalactiae, Gardnerella vaginitis, Streptococcus pyogenes, Enterococcus faecalis, Enterococcus faecium, Fusobacterium nucleatum, Porphyromonas gingivalis, Vibrio vulnificus, Clostridium botulinum, Corynebacterium diptheriae, Staphylococcus aureus, Staphylococcus epidermidis, Staphylococcus saprophyticus, Staphylococcus intermedius, Staphylococcus hyicus, Staphylococcus haemolyticus, Staphylococcus hominis, or Staphylococcus saccharolyticus.

In a particular embodiment, the bacterium is a Mycobacterium, for instance Mycobacterium species is selected from the group consisting of M. africanum, M. bovis, M. bovis BCG, M. canetti, M. caprae, M. microti, M. mungi, M. orygis, M. pinnipedii, M. suricattae, M. tuberculosis, M. avium, M. avium paratuberculosis, M. avium silvaticum, M. avium “hominissuis”, M. colombiense, M. indicus pranii, M. asiaticum, M. gordonae, M. gastri and M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. bohemicum, M. botniense, M. branderi, M. celatum, M. chimaera, M. conspicuum, M. cookie, M. doricum, M. farcinogenes, M. haemophilum, M. heckeshornense, M. intracellular, M. lacus, M. leprae, M. lepraemurium, M. lepromatosis, M. liflandii, M. malmoense, M. marinum, M. monacense, M. montefiorense, M. murale, M. nebraskense, M. saskatchewanense, M. scrofulaceum, M. shimoidei, M. szulgai, M. tusciae, M. xenopi, M. yongonense, M. intermedium, M. abscessus, M. chelonae, M. bolletii, M. fortuitum, M. fortuitum subsp. Acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodie, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense, and M. tokaiense, preferably Mycobacterium tuberculosis, Mycobacterium leprae, or Mycobacterium ulcerans.

In another preferred embodiment, the bacterium according to the invention presents a resistance to classic antibacterial drugs. The terms “antibacterial resistance”, “antibacterial agent resistance” or “antibacterial drug resistance”, as used herein, are equivalent and refer to the ability of bacteria to resist the effects of an antibacterial agent previously used to treat them. Antibacterial resistance can be defined by a decreased susceptibility to a drug through either a minimally effective, or completely ineffective, treatment response to prevent associated illnesses from a particular bacterium.

### Modulators of NEET Proteins

The NEET protein family includes three class of proteins encoded by the CISD1, CISD2 and CISD3 genes.

CISD1 gene encodes the protein mitoNEET. It was previously called C10orf70 or ZCD1 or MDS29. The gene encoding the protein is described in databases GeneCards GCID GC10P058269; HGNC: 30880; Entrez Gene: 55847; and UniGene: Hs.370102. The protein is described in UniProtKB under: Q9NZ45. Amino acid and nucleotide reference sequences of mitoNEET are disclosed in GenPept and Genbank under NP_060934.1 and NM_018464.4, respectively.

CISD2 gene encodes the protein NAF-1 (nutrient-deprivation autophagy factor-1). It was previously called WFS2 or ZCD2 and is also called Miner1, ERIS (endoplasmic reticulum intermembrane small protein) and mitoNEET related 1. The gene encoding the protein is described in databases GeneCards GCID GC04P102868; HGNC: 24212; Entrez Gene: 493856; and UniGene: Hs.444955. and Hs.745013. The protein is described in UniProtKB under: Q8N5K1. Amino acid and nucleotide reference sequences of NAF-1 are disclosed in GenPept and Genbank under NP_001008389.1 and NM_001008388.4, respectively.

CISD3 gene encodes the protein Miner2. It is also called mitoNEET-Related protein 2 or mitochondrial matrix-localized mitochondrial inner NEET protein (MiNT). The gene encoding the protein is described in databases GeneCards GCID GC17P038730; HGNC: 27578; Entrez Gene: 284106; and UniGene: Hs.713595. The protein is described in UniProtKB under ID P0C7P0. Amino acid and nucleotide reference sequences of Miner2 are disclosed in GenPept and Genbank under NP_001129970.1 and NM_001136498.1, respectively.

In one preferred embodiment, the modulator is a stabilizer or destabilizer of NEET proteins. The NEET protein modulator is able to alter the affinity of members of the protein family for its bound Fe/S cluster (increasing or decreasing).

In a first aspect, the effect of the NEET protein modulator can be measured by a binding assay, in particular by a displacement assay of a NEET protein modulator of reference on the targeted NEET protein. Such assays are well-known in the art, and for instance in the following articles (Displacement of [3H]-rosiglitazone (Geldenhuys et al, Bioorganic & Medicinal Chemistry Letters, 2010, 20(3):819-823; Geldenhuys et al, Bioorganic & Medicinal Chemistry Letters, 2011, 21(18):5498-5501)

The modulator of the NEET protein can be a modulator of at least one NEET protein selected from the group consisting of mitoNEET, NAF-1 and Miner2. In particular, it can be a modulator of a combination of NEET proteins, such as mitoNEET and NAF-1, mitoNEET and Miner2, NAF-1 and Miner2 or mitoNEET, NAF-1 and Miner2.

In a particular embodiment, NEET protein modulators are selective against one isoform of NEET protein. By “selective against” is intended herein that the modulator is more efficient for modulating one NEET protein isoform than at least one of other isoforms, preferably the two other isoforms. More preferably, selective NEET protein modulators have almost no inhibiting effect against the others, and still more preferably no inhibiting effect at all.

In a particular aspect, the NEET protein modulator does not activate PPARγ (peroxisome proliferator-activated receptor-γ).

The modulator of mitoNEET and/or NAF-1 and/or mitoNEET is a molecular compound and/or a small molecule, and/or a miRNA (for example miRNA-127 in He et al., Nature Scientific Reports 2016) and/or a siRNA (for example as supplied by Santa Cruz Biotechnologies Ref. sc-90615), and/or a mitoNEET CRISPR/Cas9 KO Plasmid (for example as supplied by Santa Cruz Biotechnologies Ref. sc-417601) and/or antisense oligonucleotides (for example as in WO2004053060) and/or an antibody (for example as in WO2004053059).

The modulator of mitoNEET and/or NAF-1 and/or Miner2 can be identified by the screening method described in WO2009026172 i.e. by crystallography combined with modelling.

In a particular embodiment, the modulator of mitoNEET and/or NAF-1 and/or Miner2 is a siRNA directed against mitoNEET and/or NAF-1 and/or Miner2.

In a preferred embodiment, NEET protein modulators are small molecules.

NEET protein modulators that can be used in the present invention, without being limited thereto, can be selected in the group consisting of Magnolol, 3,3/-di-L-tyrosine, Ac—NH-3,3′-di-L-Tyr-CO—NH2, Ac—NH-3,3′-di-L-Tyr-Gly-Gly-CO—NH2, Ac—NH-3,3′-di-L-Tyr-Ala-Ala-CO—NH, Ac—NH-3,3′-di-L-Tyr-R1-R2-6CO—NH2, Enterobactin, Cromolyn, Quercetin, Naringenin, (−)-Epicatechin, Procyanidin A2, Trans Resveratrol, Epsilon-Viniferin, Laetevirenol A, NL-1, NL-2, NL-3, NL-4, NL-5, NL-6, NL-7, NL-8, NL-9, NL-10, NL-11, NL-12, NL-14, NL-14, NL-15, NL-15, NL-16, NL-17, NL-18, NL-19, NL-20, NL-21, Resveratrol 3-sulfate, Curcumin, Kaempferol, NL-23, NL-24, NL-25, NL-26, NL-27, NL-28, NL-31, NL-32, AG104, NL-33, NL-34, Furosemide, alpha-Hydro-cinnamic acid, Glibenclamide, 7917584, 6209863, 4-Amino-1,8-naphthalimide, Triapine, Nitrofurantoin, Dantrolene, CCCP, 6636424, 6373721, 7320244, 5472855, 6634507, 5119666, 7138125, 7722368, 5472855, GSK-LSD1, Tryprostatin-A, Doxorubicin, Ursodiol, Gemfibrozil, Thiazolidinedione, Thiazolidinedione salt, Thiazolidinedione derivatives, Pioglitazone, Rosiglitazone, Rivoglitazone, Troglitazone, MSDC-0160, MSDC-0602, TT01001, MAD-28 and Resveratrol.

In a particular embodiment, the NEET protein modulator can be molecules as described in Bieganski et al. (Journal of Molecular Graphics and Modelling, 2011, 29(7):965-73), such as:

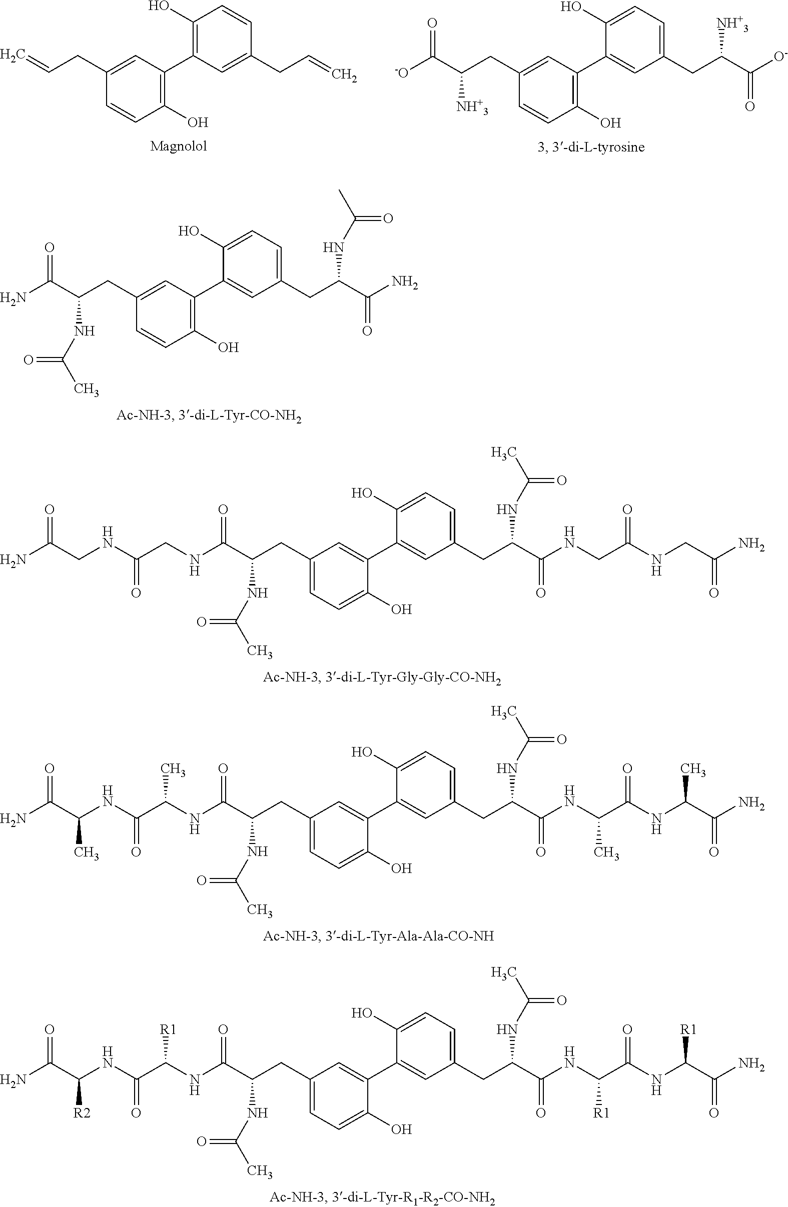

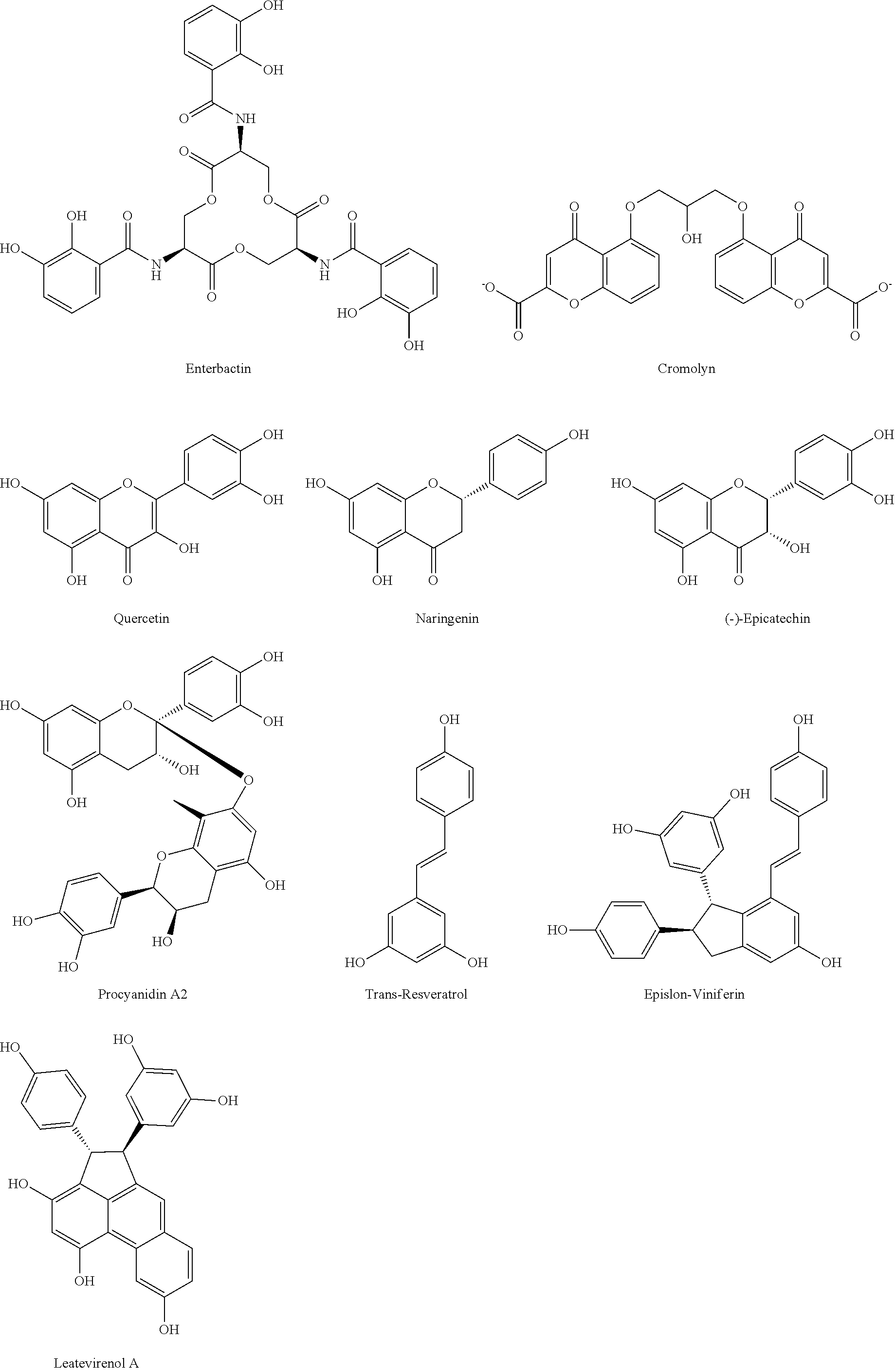

Preferably, the NEET protein modulator can be selected from the list consisting of molecules as described in Geldenhuys et al., (Bioorganic & Medicinal Chemistry Letters 26 (2016) 5350-5353), such as:

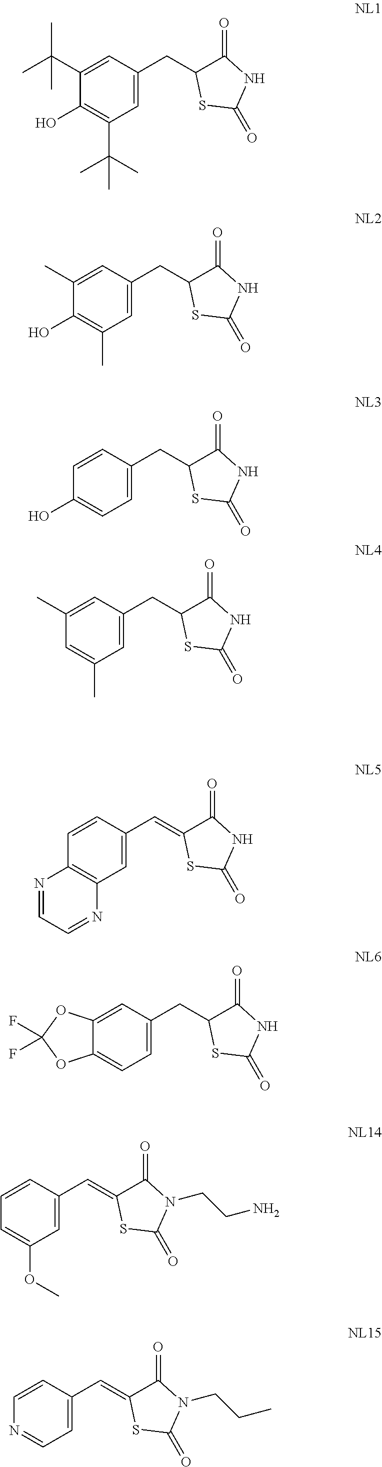

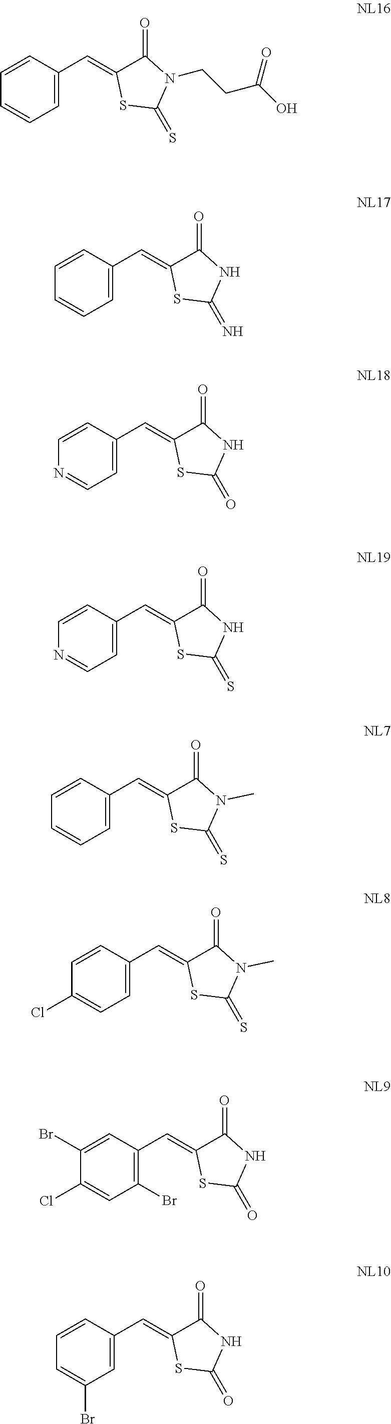

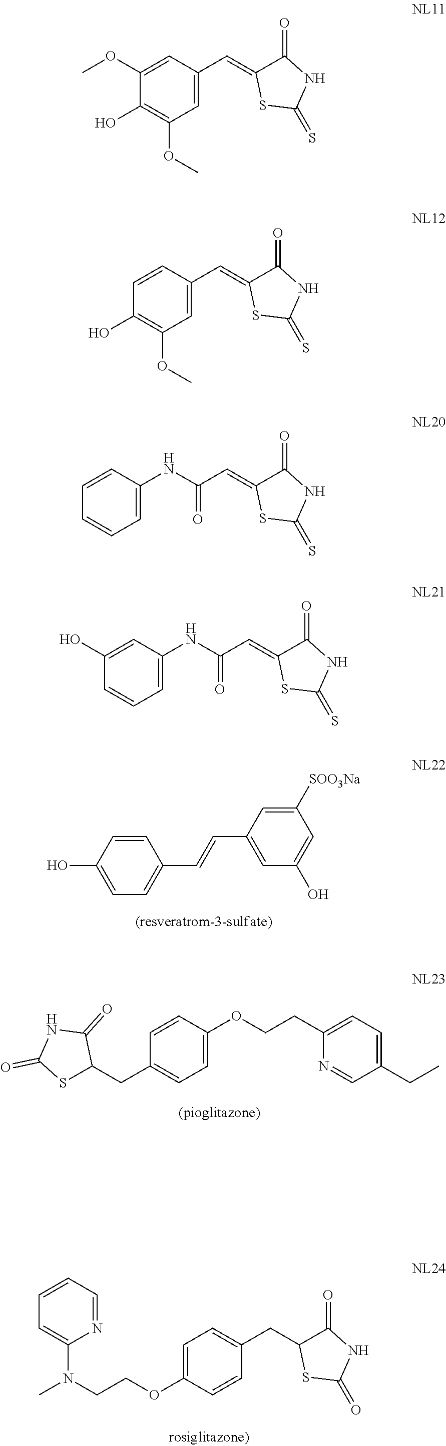

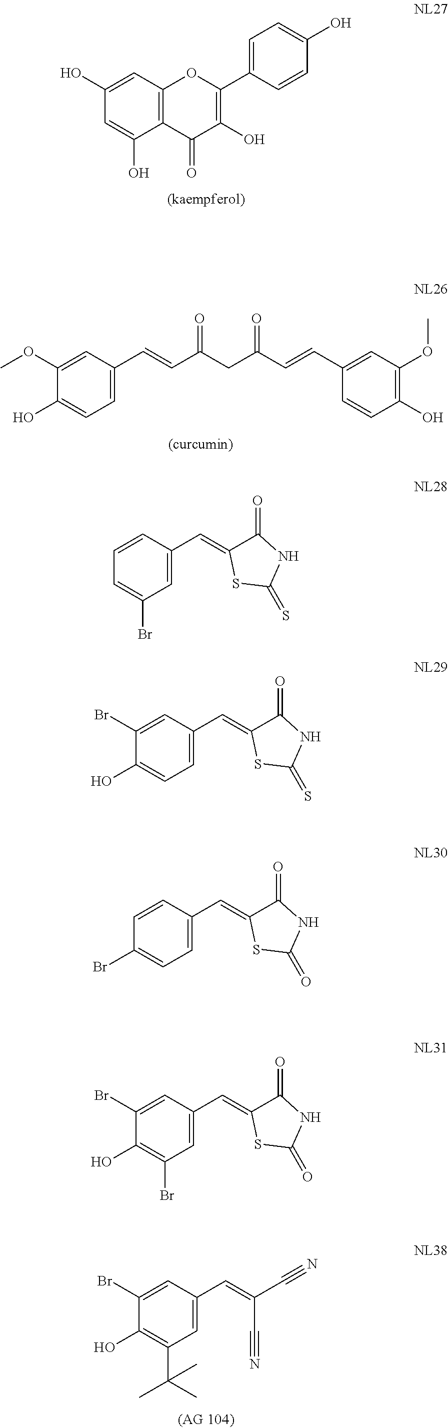

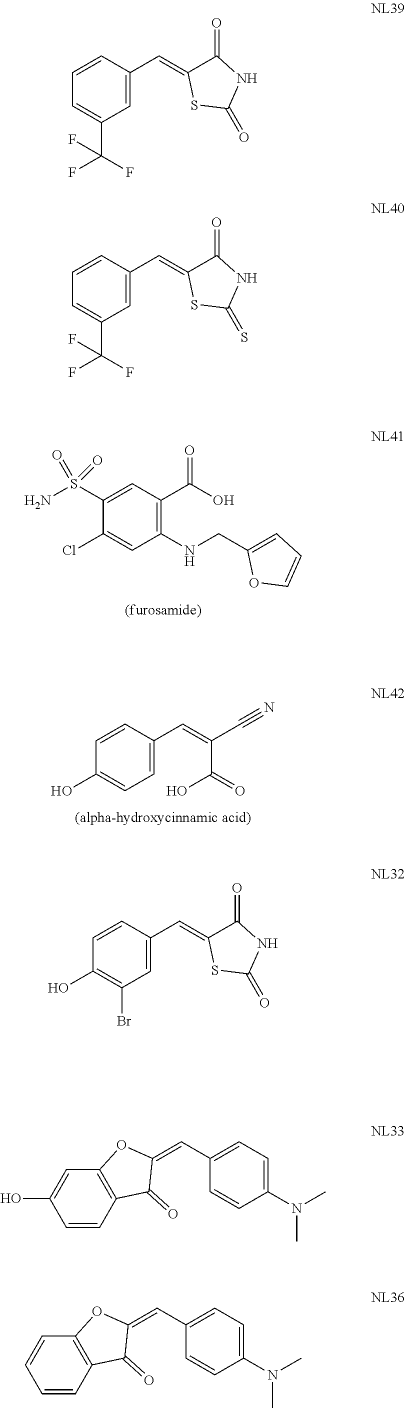

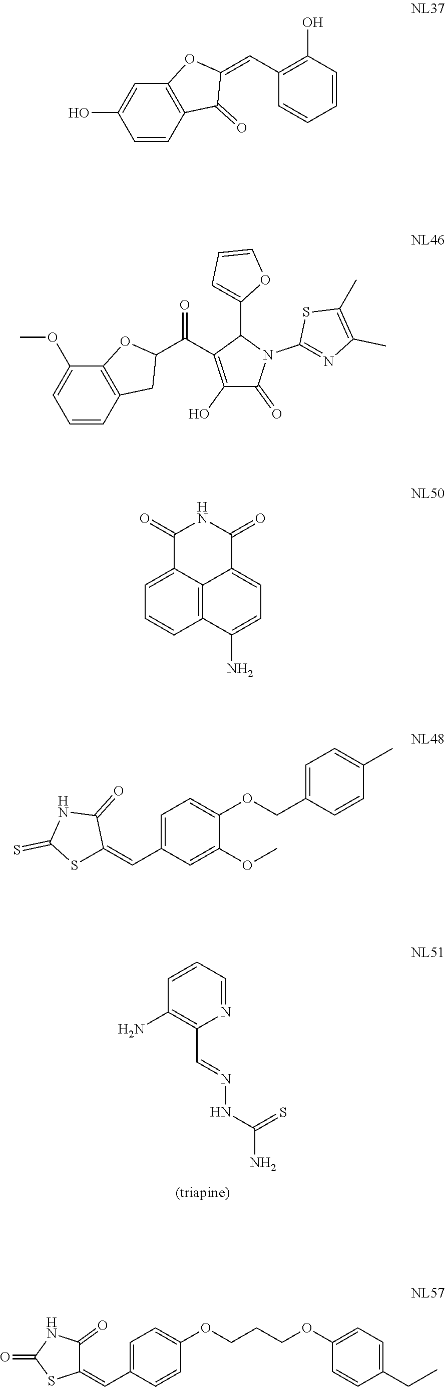

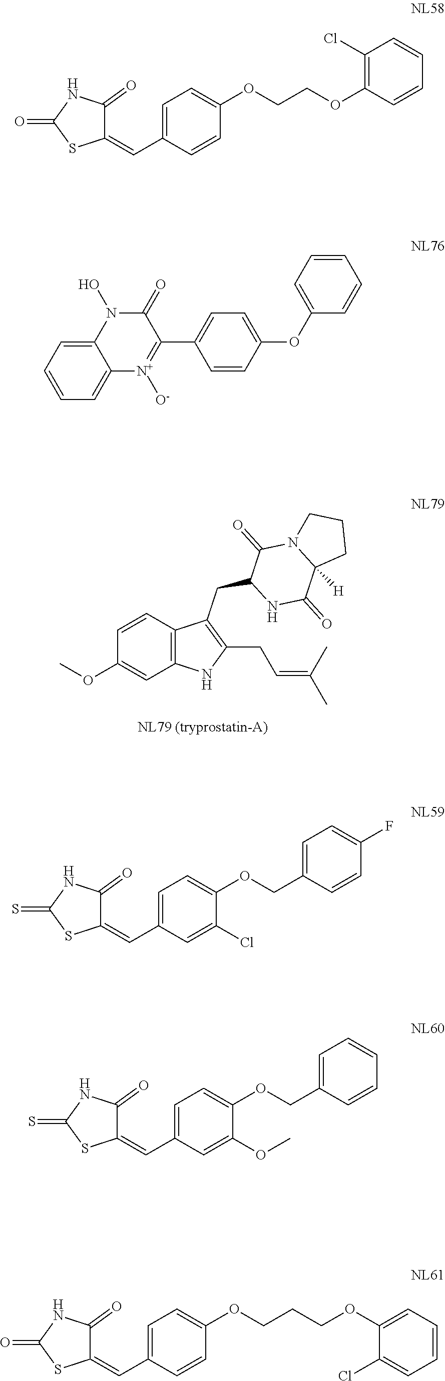

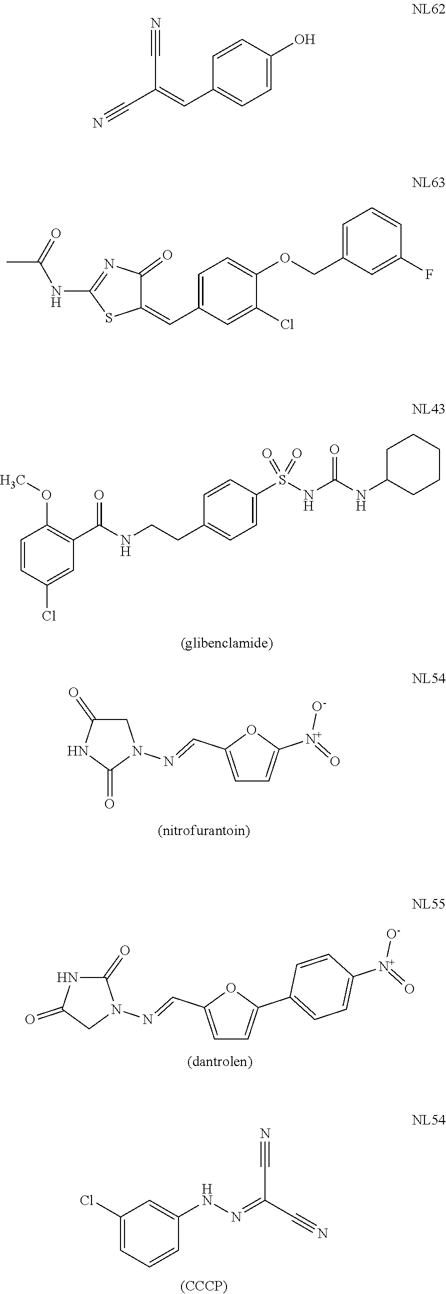

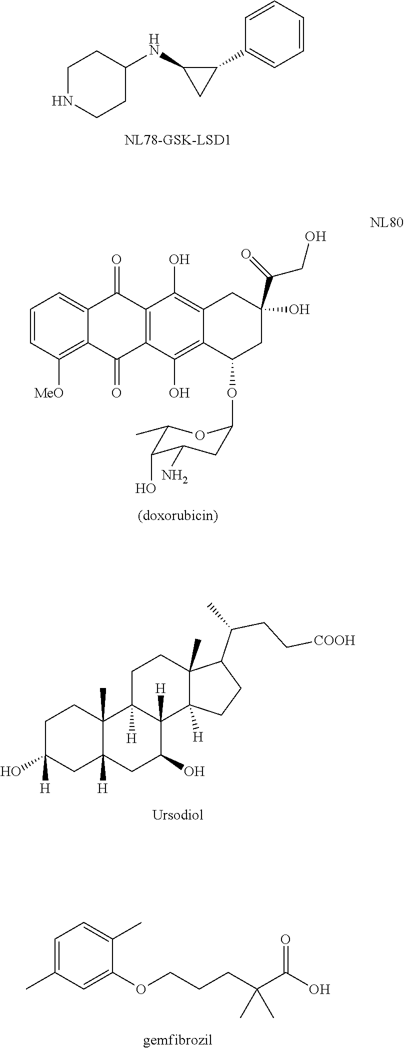

More preferably, the NEET protein modulator is the above compounds with a Ki<0.4 μM and a IC₅₀<10 μM, i.e. NL-1, Resveratrol-3-sulfate, Pioglitazone, Rosiglitazone, Curcumin, Kaempferol, 7917584, 6209863, 6636424, 6373721, 7320244, 5472855, 6634507, 5119666, GSK-LSD1 and/or Tryprostatin-A.

Even more preferably, the NEET protein modulator can be selected from the list consisting of:

a) Thiazolidinedione (TZD) derivatives such as:
Pioglitazone (CAS Registry Nb 111025-46-8), (5-[[4-(2-(5-ethylpyridin-2-yl)ethoxyphenyl]methyl]-1,3-thiazolidine-2,4-dione)

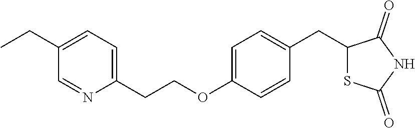 [SMILES: CCc1ccc(CCOc2ccc(CC3SC(=O)NC3=O)cc2)nc1]

Rosiglitazone (CAS Registry Nb 122320-73-4), (±)-5-[p-[2-(methyl-2-pyridylamino)ethoxy]benzyl]-2,4-thiazolidinedione

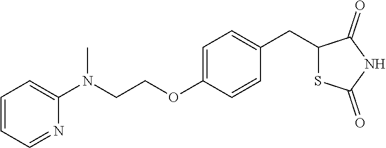 [SMILES: CN(CCOc1ccc(CC2SC(=O)NC2=O)cc1)c1ccccn1]

Rivoglitazone (CAS Registry Nb 185428-18-6), 5-[[4-[(6-methoxy-1-methylbenzimidazol-2-yl)methoxy]phenyl]methyl]-1,3-thiazolidine-2,4-dione

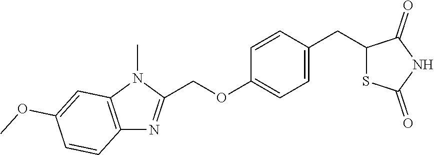 [SMILES: COc1ccc2nc(COc3ccc(CC4SC(=O)NC4=O)cc3)n(C)c2c1]

Troglitazone (CAS Registry Nb 97322-87-7), 5-[[4-[(3,4-dihydro-6-hydroxy-2,5,7,8-tetramethyl-2H-1-benzopyran-2-yl)methoxy]phenyl]methyl]-2,4-thiazlidinedione

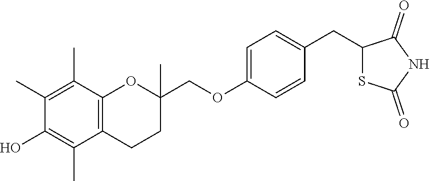 [SMILES: Cc1c(C)c2c(c(C)c1O)CCC(C)(COc1ccc(CC3SC(=O)NC3=O)cc1)O2]

MSDC-0160 (CAS Registry Nb 146062-49-9) 5-[[4-[2-(5-ethyl-2-pyridinyl)-2-oxoethoxy]phenyl]methyl]-2,4-thiazolidinedione

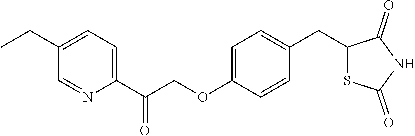 [SMILES: CCc1ccc(C(=O)COc2ccc(CC3SC(=O)NC3=O)cc2)nc1]

MSDC-0602 5-[[4-[2-(3-Methoxyphenyl)-2-oxoethoxy]phenyl]methyl]-2,4-thiazolidinedione; 5-[4-[2-(3-Methoxyphenyl)-2-oxoethoxy]benzyl]-1,3-thiazolidine-2,4-dione (CAS Registry Nb 1133819-87-0)

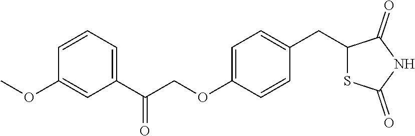 [SMILES: COc1cccc(C(=O)COc2ccc(CC3SC(=O)NC3=O)cc2)c1]

NL-1, 5-(3,5-di-tert-butyl-4-hydroxybenzyl)-4-hydroxythiazol-2(5H)-one

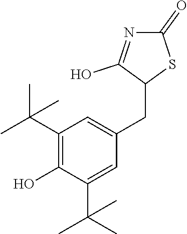 [SMILES: CC(C)(C)c1cc(CC2SC(=O)N=C2O)cc(C(C)(C)C)c1O]

In a particular embodiment, the modulator of the NEET protein can also be thiazolidinedione salts as described in US20110279657, WO2011084453, WO2011075514, WO201184456, WO2011084459, WO2014093114, WO2011133442 and WO2009038681 such as hydrogen chloride salt or dihydrogen sulfate salt and can be for example compound having the following structure:

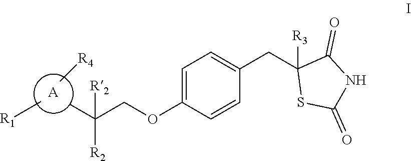

where each of R₁ and R₄ is independently selected from H, halo, aliphatic, and alkoxy, where the aliphatic or alkoxy is optionally substituted with 1-3 of halo; R′₂ is H and R₂ is H, halo, hydroxy, or optionally substituted aliphatic, —O-acyl, —O-aroyl, —O-heteroaroyl, —O(SO₂)NH₂, —O—CH(Rₘ)OC(O)Rₙ, —O—CH(Rₘ)OP(O)(ORₙ)₂, —O—P(O)(ORₙ)₂, or
each Rₘ is independently C₁₋₆ alkyl, each

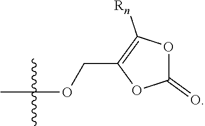

Rₙ is independently C₁₋₁₂ alkyl, C3-8 cycloalkyl, or phenyl, each of which is optionally substituted; or R₂ and R₂ together may form oxo; R₃ is H or C₁-3 alkyl; and ring A is phenyl, pyridin-2-yl, pyridin-3-yl or pyridin-4-yl, each of which is substituted with an R₁ group and an R₄ group at any chemically feasible position on ring A.
each of R₁ and R₄ can also independently selected from H, halo, aliphatic, and alkoxy, wherein the aliphatic and alkoxy are optionally substituted with 1-3 of halo;
R₂ is halo, hydroxy, or optionally substituted aliphatic, and R′₂ is H, or R₂ and R′₂ together form oxo; R₃ is H; and Ring A is phenyl as described in U.S. Pat. No. 8,304,441.

For example, such compounds can be:

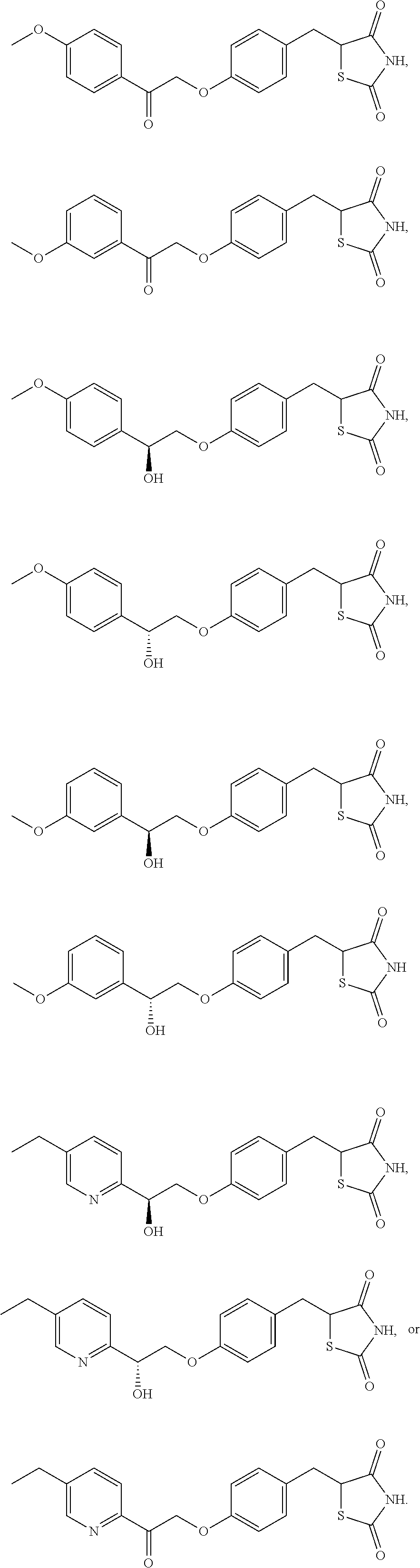

The NEET protein modulator can be any compound disclosed in US20110279657, WO2011084453, WO2011075514, WO201184456, WO2011084459, WO2014093114, WO2011133442 and WO2009038681, the disclosure of which being incorporated herein by reference.

This TDZ derivative can be synthetized by any method known by the person skilled in the art, for example according to the method disclosed in WO2011133442.

b) Resveratrol and derivatives thereof
Resveratrol

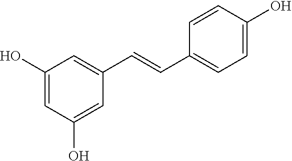 [SMILES: Oc1ccc(/C=C/c2cc(O)cc(O)c2)cc1]

Resveratrol-3-sulfate (CAS Registry Nb 858127-11-4) 5-[(1E)-2-(4-hydroxyphenyl)ethenyl]-1,3-benzenediol-1-(hydrogen sulfate), monosodium salt

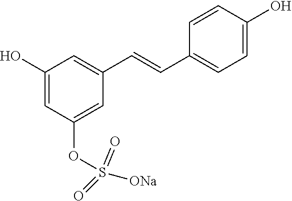

c) Derivative of cluvenone such as MAD-28

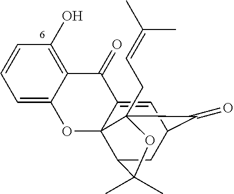 [SMILES: CC(C)=CCC12OC(C)(C)C3CC(C=C4C(=O)c5c(O)cccc5OC431)C2=O]

d) TT01001 (ethyl-4-(3-(3,5-dichlorophenyl)thioureido)piperidine-1-carboxylate)

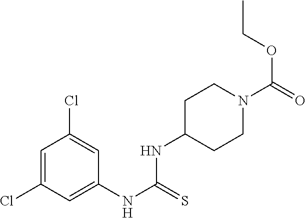 [SMILES: CCOC(=O)N1CCC(NC(=S)Nc2cc(Cl)cc(Cl)c2)CC1]

### Subject, Regimen and Administration

The subject according to the invention is an animal, preferably a mammal, or a human. However, the term “subject” can also refer to non-human animals, in particular mammals such as dogs, cats, horses, cows, pigs, sheep, donkeys, rabbits, ferrets, gerbils, hamsters, chinchillas, rats, mice, guinea pigs and non-human primates, among others, that are in need of treatment.

The human subject according to the invention may be a human at the prenatal stage, a new-born, a child, an infant, an adolescent or an adult.

In a preferred embodiment, the subject has been diagnosed with a disease. Preferably, the subject has been diagnosed with an infectious disease, especially a viral infection.

In a particular embodiment, the subject presents an antiviral resistance.

The modulator of a NEET protein or the pharmaceutical composition comprising it may be administered by any conventional route of administration. In particular, the modulator of a NEET protein or the pharmaceutical composition can be administered by a topical, enteral, oral, parenteral, intranasal, intravenous, intra-arterial, intramuscular, intratumoral, subcutaneous or intraocular administration and the like.

In particular, the modulator of a NEET protein or the pharmaceutical composition comprising it can be formulated for a topical, enteral, oral, parenteral, intranasal, intravenous, intra-arterial, intramuscular, intratumoral, subcutaneous or intraocular administration and the like.

Preferably, the modulator of a NEET protein or the pharmaceutical composition comprising it is administered by enteral or parenteral route of administration. When administered parenterally, the modulator of a NEET protein or the pharmaceutical composition is preferably administered by intravenous route of administration. When administered enterally, the modulator of a NEET protein or the pharmaceutical composition is preferably administered by oral route of administration.

The modulator of a NEET protein or the pharmaceutical composition is formulated in accordance with standard pharmaceutical practice (Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

For oral administration, the modulator of a NEET protein or the pharmaceutical composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the modulator of a NEET protein or the pharmaceutical composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The modulator of a NEET protein or the pharmaceutical composition comprising it can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Pharmaceutical compositions according to the invention may be formulated to release the active drug substantially immediately upon administration or at any predetermined time or time period after administration.

Preferably, the treatment with the modulator of a NEET protein starts no longer than a month, preferably no longer than a week, after the diagnosis of the disease of the infection by the virus. In a most preferred embodiment, the treatment starts the day of the diagnosis.

The modulator of a NEET protein or the pharmaceutical composition comprising it may be administered as a single dose or in multiple doses.

Preferably, the treatment is administered regularly, preferably between every day and every month, more preferably between every day and every two weeks, more preferably between every day and every week, even more preferably the treatment is administered every day. In a particular embodiment, the treatment is administered several times a day, preferably 2 or 3 times a day, even more preferably 3 times a day.

The duration of treatment with the modulator of a NEET protein or the pharmaceutical composition comprising it is preferably comprised between 1 day and 20 weeks, more preferably between 1 day and 10 weeks, still more preferably between 1 day and 4 weeks, even more preferably between 1 day and 2 weeks. In a particular embodiment, the duration of the treatment is of about 1 week. Alternatively, the treatment may last as long as the disease persists.

The amount of modulator of a NEET protein or the pharmaceutical composition comprising it to be administered has to be determined by standard procedure well known by those of ordinary skills in the art. Physiological data of the patient (e.g. age, size, and weight) and the routes of administration have to be taken into account to determine the appropriate dosage, so as a therapeutically effective amount will be administered to the patient.

The therapeutically effective amount of NEET modulators varies depending upon the administration mode, the age, body weight, sex and general health of the subject. It will be appreciated that there will be many ways known in the art to determine the therapeutically effective amount for a given application.

In a preferred embodiment, the total compound dose for each administration of the modulator of a NEET protein or the pharmaceutical composition comprising it is comprised between 0.00001 and 1 g, preferably between 0.01 and 10 mg.

The form of the pharmaceutical compositions, the route of administration and the dose of administration of the modulator of a NEET protein can be adjusted by the man skilled in the art according to the type and severity of the disease, and to the patient, in particular its age, weight, sex, and general physical condition.

The present invention also relates to the combined use of a modulator of a NEET protein with at least another active ingredient, preferably selected from the group consisting in an antiviral agent, an antibacterial agent, an antibiotic, an antiparasitic agent, or an antifungal agent for the treatment of infectious diseases, in particular viral diseases.

The present invention also relates to a product comprising a modulator of a NEET protein, and another active ingredient, as a combined preparation for simultaneous, separate or sequential use, in particular for use for the treatment of infectious diseases, in particular viral diseases. Preferably, the other active ingredient is selected from the group consisting in an antiviral agent, an antibacterial agent, an antibiotic, an antiparasitic agent, or an antifungal agent. Preferably, the other active ingredient is an antiviral drug.

In one embodiment, the compound of the invention can be used in combination with another antiviral drug, for instance and non-exhaustively, an agent selected from the group consisting of neuraminidase inhibitors, M2 inhibitors, RNA polymerase inhibitors, interferons (immune system modulators interferon alpha-2a and PEGylated interferon alpha-2a (Pegasys) and interferon alpha-2b (ViraferonPeg ou Introna)), antiviral vaccine, antigenic polypeptides or neutralizing antibodies directed to a viral antigenic polypeptide.

All the references cited in this application, including scientific articles and summaries, published patent applications, granted patents or any other reference, are entirely incorporated herein by reference, which includes all the results, tables, FIGURES and texts of theses references.

Although having different meanings, the terms “comprising”, “having”, “consisting in” and “containing” can be replaced one for the other in the entire application.

Further aspects and advantages of the present invention will be described in the following examples, which should be regarded as illustrative and not limiting.

### BRIEF DESCRIPTION OF FIGURES

FIG. 1: Deletion of CISD2 gene inhibits expression of the Influenza protein NP. FIG. 1 is a Western Blot with a labelling with anti-NP antibody (NP), anti-NAF-1 antibody (CISD2) or anti-actin antibody (Actin) showing the expression in a A549 cells without or with a CISD2 KO.

### EXAMPLES

### Example 1—Deletion of CISD2 Gene Inhibits Expression of the Influenza Protein NP

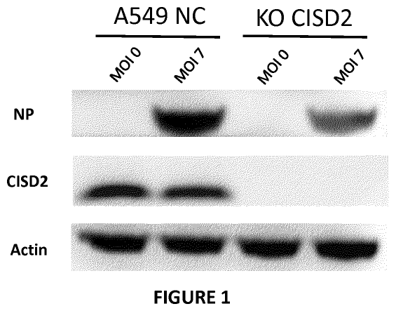

The inventors prepared a cell line with a knockout of the CISD2 gene for testing the effect of this deletion on the infection by Influenza. The results are shown in FIG. 1. The CISD2 gene knockout has a drastic effect on the expression of the viral protein NP which was significantly decreased in the KO cells.

### Materials & Methods

Construction of a CRISPR/Cas9 CISD2 Knockout Clone

To knockout the CISD2 gene, the inventors used the pLentiCRISPR V2 plasmid (Sanjana et al, 2014, Nat Methods, 11, 783-784). Oligonucleotides pairs were hybridized and cloned into the LentiCRISPR V2 vector linearized with BsmB1 to generate A549 CISD2 KO clone (oligonucleotide sequences: CACCGCAAGAAGTGCGAGTACACCA (SEQ ID NO: 1) and AAACTGGTGTACTCGCACTTCTTGC (SEQ ID NO: 2)).

To generate knockout clones, A549 cells were infected at a multiplicity infection of one with the corresponding LentiCRISPR V2 viruses and selected with puromycin (1 μg mL-1) for 5 days. Cells were then cloned in 96-well plates by limiting dilution. Isolated clones were characterized by western blot (anti-CISD2 antibody, Proteintech, 13318-1-AP).

Expression of Influenza NP Protein in A549 CSID2 KO Cells Infected by H1N1

A549 wild type and CISD2 KO cells were washed twice with DMEM and infected with the A/H1N1/New Caledonia/2006 strain at a MOI of 7 in infection medium (DMEM supplemented with 0.2 μg·ml-1 TPCK-trypsin (Sigma)) or left without virus. After 1 h at 37° C., the inoculum was discarded, and cells were washed again and incubated in DMEM with 10% FBS at 37° C. and 5% CO2.6 h post infection cells were lysed in a cold extract buffer (20 mM Tris-HCl pH 8.0, 150 mM NaCl, 1 mM EDTA, 0.5% NP40 and a protease inhibitor cocktail (Roche)). NP, CISD2 and actin were detected using standard immunoblotting techniques with anti-NP antibody (Abcam, ab128193), anti-CISD2 antibody (Proteintech, 13318-1-AP) and anti-actin antibody (Sigma, A3853).

### Example 2—Modulators of NEET Protein Inhibit Influenza Replication

Three different modulators of NEET protein (i.e., stabilisers) having different structures have been tested for their capacity to inhibit Influenza replication.

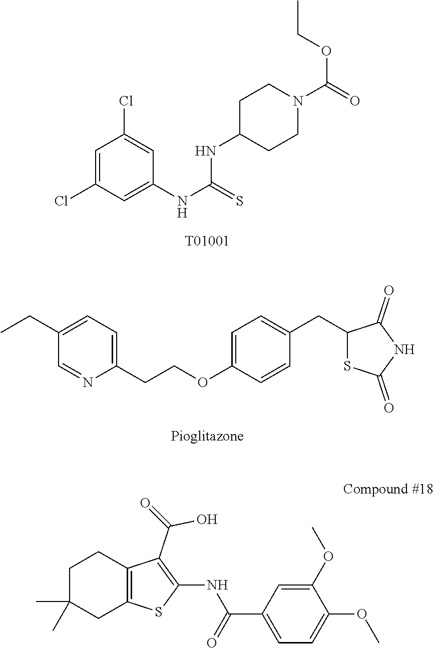

Compound #18 is a new compound that is a NEET protein modulator.

The antiviral effect of these compounds has been tested on A549 cell lines infected with H1N1 (influenza A/New Caledonia/20/99). IC50 are reported in the following Table 1. The results show that the compounds present an antiviral effect.

TABLE 1

| Compound | IC50 (μM) |
|---|---|
| T01001 | 18 |
| Pioglitazone | 38 |
| Compound #18 | 0.05 |

### Materials & Methods

Human A549 cells (80,000 cells/well in a 96 well plate) were treated with a range of concentration of test compounds and immediately infected by H1N1 A/New Caledonia/20/99 virus (clinical isolate) at MOI of 0.1 in DMEM/1% Penicillin/streptomycin supplemented with 0.25 μg/ml TPCK trypsin (Sigma) and incubated at 37° C. in 5% CO2. 48 h post-infection, supernatants (25 μl) were collected and transferred into a 96-well black flat-bottom plate, mixed with 25 μl PBS with Ca++/Mg++(Thermo Fisher) and 50 μl of 2′-(4-Methylumbelliferyl)-α-D-N-acetylneuraminic acid sodium salt hydrate stock-solution (20 μM, MUNANA, Sigma). Plates were incubated 1 h at 37° C. and reaction is stopped by adding 100 al of Stop Solution (glycine 0.1 M pH10.7/25% ethanol). The amount of fluorescent product released by MUNANA hydrolysis (4-MU) was measured in a Tecan spectrophotometer with excitation and emission wavelengths of 365 and 450 nm respectively.

### Example 3—Modulators of NEET Protein Inhibit the Replication Cycle of Other Viruses

Compound #18 was tested on other viruses. The results is shown in Table 2. The NEET protein modulators are capable of inhibiting other viruses such as West Nile Virus, Dengue and Zika with a high efficiency.

TABLE 2

| Virus | IC50 (μM) |
|---|---|
| West Nile Virus | 2.5 |
| Dengue | 5 |
| Zika | 3 |

### Materials & Methods

Assays were performed with the following strains/serotypes:
DENV serotype 2 strain New Guinea C
ZIKV strain MR766
WNV stain New-York 99

1×10⁵ Huh7 cells were infected with DENV, ZIKV, or WNV at a MOI of 0.1 pfu per cell in presence of the test compound. Two hours post-infection the inoculum was removed and cells washed twice with PBS 1×. Fresh medium containing the test compound was added. Supernatants were harvested 48 h post infection, filtered through a 0.45 am pore membrane and directly used for plaque assays.

### Plaque Assays

VeroE6 cells were infected with serial dilutions of virus supernatants. Two hours post-infection inoculum was replaced by serum-free MEM medium (Gibco, Life Technologies) containing 1.5% carboxymethyl cellulose (Sigma-Aldrich). At different days post infection (day 3 for WNV, day 4 for ZIKV, day 7 for DENV) cells were fixed by addition of formaldehyde to a final concentration of 5%. Cells were stained with crystal violet solution (1% crystal violet, 10% ethanol in H2O) for 30 min at room temperature and extensively rinsed with H2O. Infectious titers were calculated considering the corresponding dilution factor.

1-13. (canceled)

14. A method for treating an infectious disease comprising administering a modulator of a NEET protein in a subject in need thereof.

15. The method according to claim 14, wherein the infectious disease is a viral infection.

16. The method according to claim 15, wherein the viral infection is an infection by a virus selected from the group consisting of Alphaviridae, Flaviviridae, Hepadnaviridae, Herpesviridae, Orthomyxoviridae, Papovaviridae, Paramyxoviridae, Picornaviridae, Polyomaviridae, Reoviridae, Retroviridae, Rhabdoviridae, and Tobamoviruses.

17. The method according to claim 16, wherein the virus is selected from the group consisting of:
Barmah Forest virus, Middelburg virus, Ndumu virus, Bebaru virus, Chikungunya virus, Mayaro virus, O'nyong'nyong virus, Ross River virus, Semliki Forest virus, Sindbis virus, Una virus, Eastern equine encephalitis virus, Tonate virus, Venezuelan equine encephalitis virus, Cabassou virus, Everglades virus, Mosso das Pedras virus, Mucambo virus, Parmana virus, Pixuna virus, Rio Negro virus, Trocara virus, Aura virus, Babanki virus, Kyzylagach virus, Ockelbo virus, Whataroa virus, Sleeping disease virus, Samon pancreatic disease virus, Southern elephant seal virus, and Western equine encephalitis virus;
dengue virus, Hepatitis C virus, Japanese encephalitis virus, West Nile virus, yellow fever virus, Zika virus, Tick-borne encephalitis virus, Kyasanur forest disease virus, Murray Valley encephalitis virus, and Saint Louis encephalitis virus;
Hepatitis B virus;
Herpes Simplex virus 1 (HSV-1), Herpes Simplex virus 2 (HSV-2), Varicella zoster virus (VZV), Epstein-Barr virus (EBV), Cytomegalovirus (CMV), Roseolovirus (HHV-6A and 6B), HHV-7 and Kaposi's sarcoma-associated herpesvirus (KSHV);
Influenza virus A, Influenza virus B, Influenza virus C, Isavirus, Thogotovirus and Quaranjavirus;
Papillomavirus (HPC) and Polyomavirus, Simian virus 40, Merkel cell polyomavirus, Trichodysplasia spinulosa polyomavirus, BK polyomavirus, JC polyomavirus and Human polyomavirus 7;
Rubulavirus, Morbillivirus, Pneumovirus, Metapneumovirus, Avulavirus, Ferlavirus, Henipavirus, Respirovirus, mumps virus, measles virus, human parainfluenza viruses (HPIV), HPIV-1, HPIV-2, HPIV-3, HPIV-4, respiratory syncytial virus (RSV), Human respiratory syncytial virus (HRSV), canine distemper virus, phocine distemper virus, cetacean morbillivirus, Newcastle disease virus, rinderpest virus, Hendra birus and Nipah virus;
Aphthovirus, Aquamavirus, Avihepatovirus, Cardiovirus, Cosavirus, Dicipivirus, Enterovirus, Erbovirus, Hepatovirus, Kobuvirus, Megrivirus, Parechovirus, Piscevirus, Rhinovirus, Salivirus, Sapelovirus, Senecavirus, Techovirus, and Tremovirus;
Alpharetrovirus; Avian leukosis virus and Rous sarcoma virus; Betaretrovirus, Mouse mammary tumour virus; Gammaretrovirus, Murine leukemia virus and Feline leukemia virus; Deltaretrovirus, Bovine leukemia virus and Human T-lymphotropic virus; Epsilonretrovirus, Walleye dermal sarcoma virus; Lentivirus, Human immunodeficiency virus 1 and Simian, Feline immunodeficiency viruses; Spumavirus, Simian foamy virus; and
vesiculovirus, vesicular stomatitis virus, lyssavirus, rabies virus, Ephemerovirus, novirhabdovirus, cytorhabdovirus and nucleorhabdovirus.

18. The method according to claim 14, wherein the infectious disease is a bacterial infection.

19. The method according to claim 14, wherein the infectious disease is an infection by a bacterium selected from the group consisting of Helicobacter pylori, Burkholderia cepacia, Pseudomonas aeruginosa, Pseudomonas fluorescens, Pseudomonas acidovorans, Pseudomonas alcaligenes, Pseudomonas putida, Stenotrophomonas maltophilia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella parapertussis, Bordetella bronchiseptica, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Campylobacter fetus, Campylobacter jejuni, Campylobacter coli, Borrelia burgdorferi, Vibrio cholerae, Vibrio parahaemolyticus, Listeria monocytogenes, Neisseria gonorrhoeae, Neisseria meningitidis, Kingella denitrificans, Kingella indologenes, Kingella kingae, Kingella oralis, Legionella pneumophila, Moraxella bovis, Moraxella catarrhalis, Moraxella lacunata, Gardnerella vaginalis, Bacteroides fragilis, Bacteroides distasonis, Bacteroides vulgatus, Bacteroides ovalus, Bacteroides thetaiotaomicron, Bacteroides uniformis, Bacteroides eggerthii, Bacteroides splanchnicus, Clostridium difficile, Clostridium tetani, Mycobacterium species, Corynebacterium ulcerans, Streptococcus agalactiae, Gardnerella vaginitis, Streptococcus pyogenes, Enterococcus faecalis, Enterococcus faecium, Fusobacterium nucleatum, Porphyromonas gingivalis, Vibrio vulnificus, Clostridium botulinum, Corynebacterium diptheriae, Staphylococcus aureus, Staphylococcus epidermidis, Staphylococcus saprophyticus, Staphylococcus intermedius, Staphylococcus hyicus, Staphylococcus haemolyticus, Staphylococcus hominis, and Staphylococcus saccharolyticus.

20. The method according to claim 19, wherein the bacterial infection is an infection by a M. africanum, M bovis, M. bovis BCG, M. canetti, M. caprae, M. microti, M. mungi, M. orygis, M. pinnipedii, M. suricattae, M. tuberculosis, M. avium, M. avium paratuberculosis, M. avium silvaticum, M. avium “hominissuis”, M. colombiense, M. indicus pranii, M. asiaticum, M. gordonae, M. gastri and M. kansasii, M. hiberniae, M. nonchromogenicum, M. terrae, M. triviale, M. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. simiae, M. bohemicum, M. botniense, M. branderi, M. celatum, M. chimaera, M. conspicuum, M. cookie, M. doricum, M. farcinogenes, M. haemophilum, M. heckeshornense, M. intracellular, M. lacus, M. leprae, M. lepraemurium, M. lepromatosis, M. liflandii, M. malmoense, M. marinum, M. monacense, M. montefiorense, M. murale, M. nebraskense, M. saskatchewanense, M. scrofulaceum, M. shimoidei, M. szulgai, M. tusciae, M. xenopi, M. yongonense, M. intermedium, M. abscessus, M. chelonae, M. bolletii, M. fortuitum, M. fortuitum subsp. Acetamidolyticum, M. boenickei, M. peregrinum, M. porcinum, M. senegalense, M. septicum, M. neworleansense, M. houstonense, M. mucogenicum, M. mageritense, M. brisbanense, M. cosmeticum, M. parafortuitum, M. austroafricanum, M. diernhoferi, M. hodleri, M. neoaurum, M. frederiksbergense, M. aurum, M. vaccae, M. chitae, M. fallax, M. confluentis, M. flavescens, M. madagascariense, M. phlei, M. smegmatis, M. goodie, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parmense, M. phocaicum, M. poriferae, M. rhodesiae, M. seoulense, and M. tokaiense.

21. The method according to claim 14, wherein the NEET protein modulator is selected from the group consisting of a molecular compound and/or a small molecule, and/or a miRNA and/or a siRNA, and/or a mitoNEET CRISPR/Cas9 KO Plasmid, and/or antisense oligonucleotides and/or an antibody.

22. The method according to claim 14, wherein the NEET protein modulator is selected from the group consisting of Magnolol, 3,3/-di-L-tyrosine, Ac—NH-3,3′-di-L-Tyr-CO—NH2, Ac—NH-3,3′-di-L-Tyr-Gly-Gly-CO—NH2, Ac—NH-3,3′-di-L-Tyr-Ala-Ala-CO—NH, Ac—NH-3,3′-di-L-Tyr-R1-R2-6CO—NH2, Enterobactin, Cromolyn, Quercetin, Naringenin, (−)-Epicatechin, Procyanidin A2, Tran Resvertrol, Epsilon-Viniferin, Laetevirenol A, NL-1, NL-2, NL-3, NL-4, NL-5, NL-6, NL-7, NL-8, NL-9, NL-10, NL-11, NL-12, NL-14, NL-14, NL-15, NL-15, NL-16, NL-17, NL-18, NL-19, NL-20, NL-21, Resveratrol 3-sulfate, Curcumin, Kaempferol, NL-23, NL-24, NL-25, NL-26, NL-27, NL-28, NL-31, NL-32, AG104, NL-33, NL-34, Furosemide, alpha-Hydro-cinnamic acid, Glibenclamide, 7917584, 6209863, 4-Amino-1,8-naphthalimide, Triapine, Nitrofurantoin, Dantrolene, CCCP, 6636424, 6373721, 7320244, 5472855, 6634507, 5119666, 7138125, 7722368, 5472855, GSK-LSD1, Tryprostatin-A, Doxorubicin, Ursodiol, Gemfibrozil, Thiazolidinedione, Thiazolidinedione salt, Thiazolidinedione derivatives, Pioglitazone, Rosiglitazone, Rivoglitazone, Troglitazone, MSDC-0160, MSDC-0602, TT01001, MAD-28 and Resveratrol.

23. The method according to claim 14, wherein the NEET protein modulator is a thiazolidinedione (TZD) derivative selected from the group consisting of pioglitazone, rosiglitazone, troglitazone, MSDC-0160, MSDC-0602, TZD NL-1, resveratrol, resveratrol-3-sulfate, TT01001 and MAD-28.

24. The method according to claim 14, wherein the NEET protein modulator is a thiazolidinedione (TZD), salt and/or derivative thereof, selected from the group consisting of pioglitazone, rosiglitazone, Rivoglitazone, troglitazone, MSDC-0160, MSDC-0602, and NL-1.

25. The method according to claim 14, wherein the modulator is a stabiliser.

26. The method according to claim 14, wherein the NEET protein modulator is used in combination with another active ingredient.

27. The method according to claim 26, wherein said another active agent is an antiviral agent or an antibacterial agent.


SEQUENCE LISTING (2 sequences)

SEQ ID NO: 1 - DNA, 25 bp, artificial
  oligonucleotide 1
caccgcaagaagtgcgagtacacca

SEQ ID NO: 2 - DNA, 25 bp, artificial
  oligonucleotide 2
aaactggtgtactcgcacttcttgc